SEQUENCE LISTING (21 sequences)

SEQ ID NO: 1 - DNA, 22 bp, synthetic construct
  source 1..22: mol_type = other DNA
cgcagctaacgcattaaatgat

SEQ ID NO: 2 - DNA, 23 bp, synthetic construct
  source 1..23: mol_type = other DNA
ggtgctgcagttaacacattaaa

SEQ ID NO: 3 - DNA, 23 bp, synthetic construct
  source 1..23: mol_type = other DNA
ggtgtcgtagctaacgcattaaa

SEQ ID NO: 4 - DNA, 19 bp, synthetic construct
  source 1..19: mol_type = other DNA
gcgatcccytcggtagtga

SEQ ID NO: 5 - DNA, 21 bp, synthetic construct
  source 1..21: mol_type = other DNA
ggtacgggatgtatcaggatt

SEQ ID NO: 6 - DNA, 23 bp, synthetic construct
  source 1..23: mol_type = other DNA
tgtagctaacgcattaaatgatg

SEQ ID NO: 7 - DNA, 23 bp, synthetic construct
  source 1..23: mol_type = other DNA
tactaagtgtcggactaagttcg

SEQ ID NO: 8 - DNA, 21 bp, synthetic construct
  source 1..21: mol_type = other DNA
taagtgttggggaaactcagc

SEQ ID NO: 9 - DNA, 21 bp, synthetic construct
  source 1..21: mol_type = other DNA
cgcagctaacgcattaagtca

SEQ ID NO: 10 - DNA, 23 bp, synthetic construct
  source 1..23: mol_type = other DNA
tgctgcagtcaacgcattaagtt

SEQ ID NO: 11 - DNA, 22 bp, synthetic construct
  source 1..22: mol_type = other DNA
ccatctgtcacycygwtaacct

SEQ ID NO: 12 - DNA, 24 bp, synthetic construct
  source 1..24: mol_type = other DNA
caccatctgtcatwytgttaacct

SEQ ID NO: 13 - DNA, 21 bp, synthetic construct
  source 1..21: mol_type = other DNA
cacctgtcaytsggttracct

SEQ ID NO: 14 - DNA, 24 bp, synthetic construct
  source 1..24: mol_type = other DNA
caccacctgtctcaatgttaacct

SEQ ID NO: 15 - DNA, 23 bp, synthetic construct
  source 1..23: mol_type = other DNA
accacctgtacatctgttagcct

SEQ ID NO: 16 - DNA, 17 bp, synthetic construct
  source 1..17: mol_type = other DNA
cctgagtagtatgctcg

SEQ ID NO: 17 - DNA, 17 bp, synthetic construct
  source 1..17: mol_type = other DNA
cctgrgtagtacattcg

SEQ ID NO: 18 - DNA, 17 bp, synthetic construct
  source 1..17: mol_type = other DNA
cctgagtagtacgttcg

SEQ ID NO: 19 - DNA, 18 bp, synthetic construct
  source 1..18: mol_type = other DNA
cctgagtagtacgtacgc

SEQ ID NO: 20 - DNA, 257 bp, synthetic construct
  source 1..257: mol_type = other DNA
tactaagtgtcggactaagttcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggttaacattgagacaggtggtg

SEQ ID NO: 21 - DNA, 24 bp, synthetic construct
  source 1..24: mol_type = other DNA
ccctgacctacggcgtgcagtgct

(19) United States
(12) Patent Application Publication
(10) Pub. No.: US 2026/0110041 A1
(43) Pub. Date: Apr. 23, 2026

(54) PRIMER-PROBE COMBINATION AND KIT FOR RAPID BROAD-SPECTRUM DETECTION OF MYCOPLASMA, AND USE THEREOF

(71) Applicant: Wuhan Canvest Biotechnology Co., Ltd., Wuhan (CN)

(72) Inventors: Yao Hao, Wuhan (CN); Hang Luo, Wuhan (CN); Jing Xu, Wuhan (CN); Bang Zhang, Wuhan (CN); Guodong Xu, Wuhan (CN); Yujie Liu, Wuhan (CN); Xiaoying Xing, Wuhan (CN); Mingzhen Wang, Wuhan (CN)

(21) Appl. No.: 19/363,585

(22) Filed: Oct. 20, 2025

Related U.S. Application Data
(63) Continuation of application No. PCT/CN2023/127011, filed on Oct. 27, 2023.

(30) Foreign Application Priority Data
Apr. 28, 2023 (CN) 2023104803412

(51) Int. Cl. C12Q 1/689; C12Q 1/6851; C12R 1/35

(52) U.S. Cl. CPC C12Q 1/689; C12Q 1/6851; C12Q 2600/16; C12Q 2600/166; C12R 2001/35

(57) ABSTRACT

The invention discloses a primer-probe combination and a kit for the rapid broad-spectrum detection of mycoplasma, and the use thereof. The primer-probe combination comprises 10 forward primers, 5 reverse primers and 4 detection probes, wherein the forward primers have the sequences as shown in SEQ ID NOs. 1-10, the reverse primers have the sequences as shown in SEQ ID NOs. 11-15, and the detection probes have the sequences as shown in SEQ ID NOs. 16-19. The kit comprises an internal control plasmid and an internal control probe. The primer-probe combination in the kit provides broad coverage of mycoplasma types, capable of detecting over 132 species and 410 strains, with a limit of detection of 0.1-0.5 copies/μl and a sensitivity below 10 CFU/ml. It also monitors potential PCR inhibition in samples, thereby preventing false-negative results.

### FIELD OF THE DISCLOSURE

The present disclosure relates to a primer-probe combination and a kit for rapid broad-spectrum detection of mycoplasma, and use thereof.

### STATEMENT REGARDING SEQUENCE LISTING

The sequence listing associated with this application is provided in text format in lieu of a paper copy and is hereby incorporated by reference into the specification. The name of the XML file containing the sequence listing is ZL254618-UPS1.xml. The XML file is 19,769 bytes; is created on Oct. 14, 2025; and is being submitted electronically via patent center.

### BACKGROUND

Mycoplasma is a type of prokaryotic microorganism that lacks a cell wall, exhibits diverse and variable morphology, and ranges in size between bacteria and viruses (diameter 0.1-0.3 μm). To date, over 200 species of mycoplasma have been discovered in sewage, plants, animals, poultry, insects, humans, hot springs, and other high-temperature environments. The genome size of mycoplasma ranges from 0.58 to 2.2 Mbp, with a relatively low GC content (23-40 mol %). Mycoplasma can reproduce through binary fission in free-living conditions. It can also engage in symbiosis on cell membranes or parasitize intracellularly, exhibiting host cell specificity. Mycoplasma infects host cells through three main mechanisms: cell adhesion, cell invasion, and cell fusion. After being infected by mycoplasma, cells may show no obvious phenotypic changes, or they may exhibit slowed growth rates, cytopathic effects, and even chromosomal aberrations and malignant transformation. Mycoplasma-contaminated biological products can cause various diseases in humans, posing a threat to human health. The sources of mycoplasma contamination are widespread, with the highest probability of contamination coming from operating personnel and contaminated cell sources. According to reports, the contamination rate of mycoplasma in continuous cell culture lines is approximately 15-35%, while in the biopharmaceutical industry, it is about 0.44-6.70%. More than 95% of contaminations are caused by the following mycoplasma species: Mycoplasma orale (M. orale), Mycoplasma arginini (M. arginini), Mycoplasma hyorhinis (M. hyorhinis), Mycoplasma fermentans (M. fermentans), Mycoplasma hominis (M. hominis), and Acholeplasma laidlawii (A. laidlawii). Regulations and regulatory authorities in various countries explicitly require that products derived from cell cultures must be guaranteed to be free of mycoplasma contamination.

Common mycoplasma detection methods include mycoplasma culture method, indicator cell method, electron microscopy, immunoassay, biochemical assay, and nucleic acid amplification testing (NAT). Currently, the methods universally adopted by pharmacopoeias are the mycoplasma culture method and the indicator cell culture method. The mycoplasma culture method, also known as the 28-day culture method, is the most classical approach for isolating and detecting mycoplasma. Its sensitivity can reach 1-10 CFU/mL. However, its disadvantages include a long cultivation time, the need for various culture media to detect different mycoplasma species, and the potential for missing detection of fastidious mycoplasma species (such as M. hyorhinis). The indicator cell culture method offers a time advantage (3-5 days) compared to the mycoplasma culture method but has lower sensitivity. It can only detect cell-adhering mycoplasmas and is prone to false-negative results with poorly adherent species like M. orale and M. arginini. Furthermore, it is not suitable for virus samples that can cause cytopathic effects in the indicator cells. Both the mycoplasma culture method and the indicator cell culture method are unsuitable for the rapid testing of cell products with high timeliness requirements, such as chimeric antigen receptor T (CAR-T) cells.

NAT is a detection method based on the amplification of mycoplasma nucleic acid sequences (DNA/RNA). It can be classified into RT-PCR, PCR, Nested-PCR, Touch-down PCR, qPCR, and isothermal amplification. Due to its convenience, speed, high sensitivity, and good specificity, NAT has been adopted by the european pharmacopoeia and the japanese pharmacopoeia. The United States Pharmacopeia also explicitly states that validated NAT or methods relying on enzyme activity can be used for mycoplasma testing, provided that the new method demonstrates comparability to the pharmacopeial method. The advantages of qPCR include: the ability to accurately quantify the initial template amount via the cycle threshold (Ct) value; support for high-throughput and multiplex detection within a single reaction well; and excellent performance in terms of specificity, sensitivity, accuracy, and the breadth of detectable species. Consequently, qPCR is widely used for quality control testing of biological products both domestically and internationally.

### SUMMARY

To address the limitations of the mycoplasma culture method and the indicator cell culture method for rapid testing of biological products, particularly time-sensitive cell-based products, the present disclosure provides a primer-probe combination, a kit, and their application for rapid and broad-spectrum detection of mycoplasma. Given the wide variety of mycoplasma species, designing multiple primer-probe pairs targeting relatively conserved sequences holds promise for achieving simultaneous and rapid detection of multiple mycoplasma types within a single qPCR reaction system. This approach can facilitate the rapid testing and release of biological products, especially time-sensitive cell-based products. The solution of the present disclosure enables the detection of hundreds of mycoplasma species in biological products within 4 hours, offering extensive coverage (>132 species, >410 strains), high specificity (no cross-reactivity with bacteria, fungi, human and animal cells, or viruses), superior sensitivity (LOD of 0.1-0.5 copies/pl, equivalent to 1-5 copies/reaction; mycoplasma sensitivity below 10 CFU/mL), and strong robustness (resistance to matrix interference, stability unaffected by repeated freeze-thaw cycles). It meets the mycoplasma NAT requirements of the european and japanese pharmacopoeias and is capable of serving as an alternative or supplement to traditional mycoplasma detection methods. The purpose of the disclosure is realized through the following technical solutions.

A primer-probe combination for rapid and broad-spectrum detection of mycoplasma, comprising 10 forward primers, 5 reverse primers, and 4 detection probes; wherein the nucleotide sequences of the 10 forward primers are set forth in SEQ ID NO: 1-10, the nucleotide sequences of the 5 reverse primers are set forth in SEQ ID NOs: 11-15, and the nucleotide sequences of the 4 detection probes are set forth in SEQ ID NOs: 16-19; and the 5′ end of each detection probe is conjugated to a first fluorescent label and the 3′ end is conjugated to a quencher.

In the nucleotide sequences provided in the present disclosure, the degenerate base W represents bases A or T, the degenerate base Y represents bases C or T, the degenerate base S represents bases G or C, and the degenerate base R represents bases A or G.

Preferably, the first fluorescent label is selected from the group consisting of FAM, TET, NED, ROX, CY3, CY5, VIC, JOE, and HEX; and the quencher is selected from the group consisting of MGB, TAMRA, NFQ, ECLIPSE, DABCYL, BHQ1, and BHQ2.

In a preferred embodiment, the first fluorescent label is FAM, and the quencher is MGB.

The present disclosure also provides the use of the aforementioned primer-probe combination in the preparation of a product for the rapid and broad-spectrum detection of mycoplasma.

A kit for rapid and broad-spectrum detection of mycoplasma, comprising the aforementioned primer-probe combination, and further including an internal control plasmid and a corresponding internal control probe. The internal control plasmid and probe are used to monitor PCR inhibition in the test sample during mycoplasma detection. Typically, the internal control plasmid is stored in a buffer solution; for example, in one embodiment, it is stored in TE buffer at a concentration of 300 copies/pl.

Preferably, the internal control plasmid contains a target fragment with the nucleotide sequence set forth in SEQ ID NO: 20, and the internal control probe has the nucleotide sequence set forth in SEQ ID NO: 21. The 5′ end of the internal control probe is conjugated to a second fluorescent label that is different from the first fluorescent label, and its 3′ end is conjugated to a quencher, which may be selected from the group consisting of MGB, TAMRA, NFQ, ECLIPSE, DABCYL, BHQ1, and BHQ2.

Preferably, the kit also includes a qPCR reaction mix, a positive control (PC), and PCR-grade water.

More specifically, the qPCR reaction mix is formulated to contain a PCR buffer, dNTPs, a hot-start Taq polymerase, and MgCl₂. The PC is provided as a buffer containing the genomic DNA of Mycoplasma pneunoniae (M. pneunoniae) and M. orale.

In a preferred embodiment, PC is prepared by formulating the genomic DNA of M. pneumoniae and M. orale in TE buffer at a concentration of 100 copies/pl. PCR-grade water may be selected as nuclease-free water. All the aforementioned reagents in the kit are preferably stored at −20° C., and repeated freeze-thaw cycles should be minimized.

A method for rapid and broad-spectrum detection of mycoplasma, not intended for disease diagnosis or treatment, utilizes the kit provided by the present disclosure to qualitatively detect mycoplasma contamination in sampled specimens and biological products (including in-process and final products). Biological products include cell banks, virus seed stocks, gene therapy products, etc. Sampled specimens encompass samples obtained through various collection pathways, such as human tissues and blood samples.

The kit of the present disclosure detects a broad panel of mycoplasma, totaling >132 species and >410 strains, as detailed in table 1.

TABLE 1

| Mollicutes | NO. of Species | NO. of Strains |
|---|---|---|
| Mycoplasma sp. | 101 | 328 |
| Spiroplasma sp. | 14 | 35 |
| Ureaplasma sp. | 5 | 28 |
| Entomoplasma sp. | 4 | 5 |
| Acholeplasma sp. | 8 | 14 |
| Total | 132 | 410 |

Preferably, the method for rapid and broad-spectrum detection of mycoplasma comprises the following steps:
S1. Sample preparation and nucleic acid extraction.

Prepare the test sample to form a test solution, and spike the internal control plasmid into either the test solution or the qPCR reaction mix. Prepare a negative control (NEC) by diluting the internal control plasmid in sterile saline (0.9% sterile NaCl solution). Prepare PC by adding the positive control genome to a mixture of the internal control plasmid and nuclease-free water, followed by dilution. Prepare a no-template control (NTC) by diluting the internal control plasmid in nuclease-free water. Extract nucleic acids from both the test solution and NEC.
S2. qPCR setup and amplification.

Create a dual-channel setup on a real-time PCR instrument for the first fluorescent label and the second fluorescent label. Perform qPCR reactions separately for the test solution, NEC, PC, and NTC. Record the Ct values for both channels from each reaction.
S3. Result Interpretation based on Ct value.

Mycoplasma Positive: The Ct value in the mycoplasma channel (first fluorescent label) is <40, with a normal amplification curve.

PCR Inhibition: No Ct value is obtained in both the mycoplasma channel and the internal control channel (second fluorescent label).

Mycoplasma Negative: No Ct value is obtained in the mycoplasma channel, while the Ct value in the internal control channel is <40 with a normal amplification curve.

Invalid Result: The Ct value in the mycoplasma channel is 40 with a normal amplification curve, and the Ct value in the internal control channel is <40 with a normal amplification curve.

PCR Inhibition: The Ct value in the mycoplasma channel is 40 with a normal amplification curve, but no Ct value is obtained in the internal control channel.

In step S1, the internal control plasmid may be added directly to the test solution-that is, before the nucleic acid extraction from the test sample, or it may be added to the qPCR reaction mix prior to the qPCR amplification-that is, after the nucleic acid extraction. Both methods of adding the internal control plasmid fall within the scope of protection of the present disclosure.

When the internal control plasmid is added directly to the test solution, the test sample is first centrifuged to pellet the cells, and the internal control plasmid may then be incorporated using any one of the following four methods:
(1) The supernatant is directly collected, and the internal control plasmid is added to formulate the test solution.
(2) The supernatant is collected and centrifuged again to enrich mycoplasma. The resulting pellet is resuspended after supernatant removal, and the internal control plasmid is added to formulate the test solution.
(3) The supernatant is collected and used to resuspend the pelleted cells (containing approximately 10\hat{ }6 cells), and the internal control plasmid is then added to formulate the test solution.
(4) The supernatant is collected and centrifuged again to enrich mycoplasma. The mycoplasma pellet is resuspended after supernatant removal. Then use this resuspension to resuspend the original cell pellet, and the internal control plasmid is added to formulate the test solution.

When choosing to add the internal control plasmid to the qPCR reaction mix, the key difference from the four methods described above is as follows: the internal control plasmid is not added during the preparation of the test solution. Instead, a quantified amount of the internal control plasmid is thoroughly mixed with the qPCR reaction mix to prepare a mixture. Prior to the qPCR amplification, a specified volume of this mixture is combined with the nucleic acid template. The reaction is then performed, typically in several replicates.

The specific methods for adding the internal control plasmid as described above also fall within the scope of protection of the present disclosure.

Taking cell culture as an example, the method for rapid and broad-spectrum detection of mycoplasma specifically comprises the following steps:
S1. Preparation of the test solution (When internal control plasmid is added before nucleic acid extraction).

Transfer 1 ml of cell culture suspension or culture supernatant to a 1.5 ml EP tube. Centrifuge at 500×g for 5 minutes to pellet the cells. Then, transfer 200 μl of the resulting supernatant to a new 1.5 ml EP tube. Add 5 μl of the internal control plasmid to this supernatant to prepare the test solution.

Or, transfer 1 ml of cell culture suspension or culture supernatant to a 1.5 ml EP tube and centrifuge at 500×g for 5 minutes to pellet the cells. Transfer 1 ml of the cleared supernatant (after cell pellet removal) to another 1.5 ml EP tube. Centrifuge this supernatant at 20,000×g for 10 minutes at 4° C. to enrich mycoplasma. Carefully removal the supernatant (approximately 20-30 pl of supernatant can be left behind to avoid loss of the mycoplasma). Resuspend the mycoplasma pellet in 170 μl of 0.9% sterile NaCl solution. Add 5 μl of the internal control plasmid to this suspension to prepare the test solution.

Or, transfer 1 ml of cell culture suspension or culture supernatant to a 1.5 ml EP tube. Centrifuge at 500×g for 5 minutes to pellet the cells. Then, take 200 μl of the supernatant to resuspend the cell pellet (containing approximately 10\hat{ }6 cells). Add 5 μl of the internal control plasmid to this mixture to prepare the test solution.

Or, transfer 1 ml of cell culture suspension or culture supernatant to a 1.5 ml EP tube and centrifuge at 500×g for 5 minutes to pellet the cells. Transfer 1 ml of the cleared supernatant (after cell pellet removal) to another 1.5 ml EP tube. Centrifuge at 20,000×g for 10 minutes at 4° C. to enrich mycoplasma. Carefully aspirate the supernatant (leaving approximately 20-30 pl behind). Resuspend the mycoplasma pellet in 170 μl of 0.9% sterile NaCl solution. Use this resuspension to resuspend the original cell pellet (containing approximately 10\hat{ }6 cells). Finally, add 5 μl of the internal control plasmid to prepare the test solution.

Add 5 μl of the internal control plasmid to 195 μl of 0.9% sterile NaCl solution to prepare NEC.

Extract nucleic acids from both the test solution and NEC using the QIAamp MinElute Virus Spin Kit (QIAGEN, 57704). Elute the nucleic acids using 100 μl of RNase-free water preheated to 65° C., then reload the eluate onto the spin column and perform a second elution step. This process yields the sample template and the negative control template, respectively.

Prepare PC by combining 0.5 pl of the positive control genome, 0.5 pl of the internal control plasmid, and 9 μl of nuclease-free water.

Prepare NTC by adding 0.5 pl of the internal control plasmid to 9.5 pl of nuclease-free water.
S2. Preparation of PCR tubes (Perform this procedure on a cooling ice box).

Test sample tube: Combine 15 μl of qPCR reaction mix with 10 μl of the sample template in a reaction tube. Prepare 2 replicates.

NEC tube: Combine 15 μl of qPCR reaction mix with 10 μl of the negative control template in a reaction tube. Prepare 2 replicates.

PC tube: Combine 15 μl of qPCR reaction mix with 10 μl of PC in a reaction tube. Prepare 2 replicates.

NTC tube: Combine 15 μl of qPCR reaction mix with 10 μl of NTC in a reaction tube. Prepare 2 replicates.

Create a dual-channel setup for the first fluorescent label (e.g., FAM) and the second fluorescent label (e.g., HEX) on the real-time PCR instrumen, then perform qPCR reactions separately for the test sample tubes, NEC tubes, PC tubes, and NTC tubes to acquire the Ct value for both channels. The qPCR reaction conditions are as follows: 37° C. for 2 minutes; 95° C. for 5 minutes; followed by 45 cycles of 95° C. for 15 seconds, 53° C. for 25 seconds, and 72° C. for 20 seconds.
S3. Perform result interpretation based on the Ct value.

Select the Ct value reading mode according to the specific qPCR instrument and its accompanying control software. Using the BIO-RAD CFX96 as an example, choose the “Regression” mode for the Cq Determination Mode, or alternatively, select the “Single Threshold” mode and manually drag the threshold line to the starting point of the linear amplification phase of PC. Record the Ct values for the test samples, NEC, PC, and NTC. The expected results of control samples should correspond to those outlined in table 2.

TABLE 2

| Control | | |
|---|---|---|
| Samples | Mycoplasma Channel (FAM) | Internal Control Channel (HEX) |
| PC | Positive (Ct < 40, with a | Positive (Ct < 40, with a normal |
| | normal amplification curve) | amplification curve) |
| NEC | Negative (no Ct value or no | Positive (Ct < 40, with a normal |
| | normal amplification curve) | amplification curve) |
| NTC | Negative (no Ct value or no | Positive (Ct < 40, with a normal |
| | normal amplification curve) | amplification curve) |

Interpretation of the test sample results is based on the principles given in table 3

TABLE 3

| Mycoplasma Channel | Internal Control Channel | |
|---|---|---|
| (FAM) | (HEX) | Result |
| Positive (Ct < 40, with a | Irrelevance | Mycoplasma Positive |
| normal amplification curve) | | |
| Negative (no Ct value) | Negative (no Ct value) | PCR Inhibition |
| Negative (no Ct value) | Positive (Ct < 40, with a | Mycoplasma Negative |
| | normal amplification curve) | |
| Borderline result (Ct ≥ 40, | Positive (Ct < 40, with a | Invalid Result; the test |
| with a normal amplification | normal amplification curve) | shoud be repeated with |
| curve) | | an increased extraction |
| | | amount |
| Borderline result (Ct ≥ 40, | Negative (no Ct value) | PCR Inhibition |
| with a normal amplification | | |
| curve) | | |

It should be noted that the preparation method described in step S2 above is based on the premise that the internal control plasmid has already been added to the test solution in step S1. Under this condition, the difference in Ct value between the internal control channel in a mycoplasma-negative sample and the NEC must be within ±3 cycles.

If the internal control plasmid is added to the qPCR reaction mix, the procedure for step S2 can be modified as follows: mix 15 μl of qPCR reaction mix with 0.5 μl of internal control plasmid, and then combine 15 μl of the resulting mixture with 10 μl of nucleic acid template. Under this condition, the difference in Ct value between the internal control channel in a mycoplasma-negative sample and the NEC must be within ±2 cycles.

In summary, the method for rapid and broad-spectrum detection of mycoplasma provided by the present disclosure offers multiple options for incorporating the internal control plasmid. Both addition methods described above fall within the scope of protection of this disclosure.

The advantages of the technical scheme proposed in the disclosure are:
1. Compared to traditional mycoplasma detection methods, a significant advantage of this kit and its associated method in this disclosure is its operational simplicity, short turnaround time, and low sample volume requirement. It provides support for early-stage mycoplasma contamination monitoring and rapid release of biological products, particularly cell therapy products.
2. Compared to commonly available commercial PCR-based rapid mycoplasma tests, the primer-probe combination in this disclosure offers broad coverage, capable of detecting over 132 species and 410 strains of mycoplasma. It exhibits high specificity, showing no cross-reactivity with bacteria, fungi, human or animal cells, or common viruses. Simultaneously, it possesses high sensitivity with a limit of detection (LOD) of 0.1-0.5 copies/pl (equivalent to 1-5 copies/reaction) and a sensitivity below 10 CFU/ml. Its robustness is strong, meeting the requirements for mycoplasma NAT outlined in the european and japanese pharmacopoeias.
3. The kit of the present disclosure includes an internal control system (comprising the internal control plasmid and probe), which monitors extraction efficiency and potential PCR inhibition in test samples, thereby preventing false-negative results and ensuring more accurate and reliable outcomes.

### BRIEF DESCRIPTION OF THE DRAWINGS

Accompanying drawings provide a further understanding of embodiments of the disclosure. The drawings form a part of the disclosure and illustrate the principle of the embodiments of the disclosure along with the literal description. Apparently, the drawings in the description below are merely some embodiments of the disclosure. A person skilled in art can obtain other drawings according to these drawings without creative efforts. In the figures:

FIGS. 1A-1J show the standard curves for real-time quantitative fluorescent amplification in example 2;

FIG. 2 shows the verification results of cross-reactivity between the primer-probe combination and non-mycoplasma species in example 2.

FIG. 3 shows the verification results of cross-reactivity between the primer-probe combination and the internal control plasmid in example 2.

FIG. 4 shows the specificity validation results of the internal control primers and probes in example 3.

FIG. 5 shows the impact of adding the internal control plasmid on the mycoplasma detection system in example 3.

FIGS. 6A-6J show the verification results of cell matrix interference on the amplification curve of mycoplasma standards in example 6.

### DETAILED DESCRIPTION OF PREFERRED EMBODIMENTS

The invention will be further described in detail in combination with embodiments to make the purpose, technical scheme, and advantages of the invention clear. The specific embodiments described herein are only used to explain the invention and are not intended to limit the invention.

Unless otherwise defined, all technical and scientific terms used in the disclosure have the same meanings as those commonly understood by those skilled in the art belonging to the disclosure.

In the present invention, the term “mycoplasma” is not limited to the genus Mycoplasma, but is interpreted broadly to refer to the class Mollicutes, encompassing genera such as Mycoplasma, Ureaplasma, Asteroleplasma, Spiroplasma, Entomoplasma, Mesoplasma, Anaerobicoplasma, among others.

The primer-probe combination provided in a embodiment of the present disclosure comprises 10 forward primers, 5 reverse primers, and 4 detection probes.

The kit provided in a embodiment of the present disclosure includes, in addition to the aforementioned primer-probe combination, an internal control plasmid and a corresponding internal control probe for monitoring PCR inhibition in the test sample during detection.

It should be noted that a fluorescent label is conjugated to the 5′ end of both the detection probes and the internal control probe. The fluorescent label may be selected from the group consisting of FAM, TET, NED, ROX, CY3, CY5, VIC, JOE, and HEX. To achieve the detection purpose, it is only necessary to ensure that the fluorescent labels of the detection probe and the internal control probe are different. For example, in a embodiment of the present disclosure, the detection probe has a FAM fluorescent label conjugated to its 5′ end and an MGB quencher conjugated to its 3′ end, while the internal control probe has a HEX fluorescent label conjugated to its 5′ end and a BHQ1 quencher conjugated to its 3′ end.

In the following examples, all nucleotide sequences, including primers, probes, the internal control plasmid, and target fragments, were synthesized by Wuhan Tianyi Huiyuan Biotechnology Co., Ltd., unless otherwise specified. All conventional reagents, such as the qPCR reaction buffer, were commercially available products from Nanjing Vazyme Biotech Co., Ltd.

### Example 1

This example provides a primer-probe combination for the broad-spectrum detection of mycoplasma, along with an internal control plasmid and its corresponding probe.
1. Design of the primer-probe combination.

The vast number of mycoplasma species, their varying degrees of phylogenetic relatedness, and the significant divergence in their genomic sequences preclude the use of a single primer-probe pair for universal detection. This example selected the following 18 mycoplasma species based on those stipulated in the european pharmacopoeia and those that are relatively common, pathogenic in routine production: Mycoplasma gallisepticum (M. gallisepticum), Mycoplasma pirum (M. pirum), M. pneumoniae, Mycoplasma genitalium (M. genitalium), Mycoplasma penetrans (M. penetrans), Ureaplasma urealyticum (U. urealyticum), Spiroplasma citri (S. citri), Mycoplasma mycoides (M. mycoides), M. arginini, M. hominis, Mycoplasma arthritidis (M. arthritidis), M. orale, Mycoplasma salivarium (M. salivarium), M. hyorhinis, M. fermentans, Mycoplasm synoviae (M. synoviae), Mycoplasma bovis (M. bovis), and A. laidlawii. Using these 18 species as templates, their sequences were aligned against the 168 rRNA genes of bacteria and the 188 rRNA genes of fungi and animal cells. Subsequently, a combination of 10 forward primers, 5 reverse primers, and 4 detection probes targeting the mycoplasma 168 rRNA gene were designed.

The sequences of the 10 forward primers are as follows:

FP1:
(SEQ ID NO: 1)
CGCAGCTAACGCATTAAATGAT;
FP2:
(SEQ ID NO: 2)
GGTGCTGCAGTTAACACATTAAA;
FP3:
(SEQ ID NO: 3)
GGTGTCGTAGCTAACGCATTAAA;
FP4:
(SEQ ID NO: 4)
GCGATCCCYTCGGTAGTGA;
FP5:
(SEQ ID NO: 5)
GGTACGGGATGTATCAGGATT;
FP6:
(SEQ ID NO: 6)
TGTAGCTAACGCATTAAATGATG;
FP7:
(SEQ ID NO: 7)
TACTAAGTGTCGGACTAAGTTCG;
FP8:
(SEQ ID NO: 8)
TAAGTGTTGGGGAAACTCAGC;
FP9:
(SEQ ID NO: 9)
CGCAGCTAACGCATTAAGTCA;
FP10:
(SEQ ID NO: 10)
TGCTGCAGTCAACGCATTAAGTT

The sequences of the 5 reverse primers are as follows:

RP1:
(SEQ ID NO: 11)
CCATCTGTCACYCYGWTAACCT;
RP2:
(SEQ ID NO: 12)
CACCATCTGTCATWYTGTTAACCT;
RP3:
(SEQ ID NO: 13)
CACCTGTCAYTSGGTTRACCT;
RP4:
(SEQ ID NO: 14)
CACCACCTGTCTCAATGTTAACCT;
RP5:
(SEQ ID NO: 15)
ACCACCTGTACATCTGTTAGCCT.

The sequences of the 4 detection probes are as follows:

Probe1:
(SEQ ID NO: 16)
CCTGAGTAGTATGCTCG;
Probe2:
(SEQ ID NO: 17)
CCTGRGTAGTACATTCG;
Probe3:
(SEQ ID NO: 18)
CCTGAGTAGTACGTTCG;
Probe4:
(SEQ ID NO: 19)
CCTGAGTAGTACGTACGC.

The detection probes have a FAM fluorescent label conjugated to their 5′ end and an MGB quencher conjugated to their 3′ end.
2. Design of the internal control plasmid and probe.

A target fragment for the internal control plasmid was artificially synthesized. The nucleotide sequence of this target fragment is as follows:

(SEQ ID NO: 20)
TACTAAGTGTCGGACTAAGTTCGTGACCACCCTGACCTACGGCGTG
CAGTGCTTCAGCCGCTACCCCGACCACATGAAGCAGCACGACTTC
TTCAAGTCCGCCATGCCCGAAGGCTACGTCCAGGAGCGCACCATC
TTCTTCAAGGACGACGGCAACTACAAGACCCGCGCCGAGGTGAAG
TTCGAGGGCGACACCCTGGTGAACCGCATCGAGCTGAAGGGCATC
GACTTCAAGGTTAACATTGAGACAGGTGGTG.

The target fragment was cloned into the pGEM-T easy plasmid. The resulting recombinant plasmid serves as the internal control plasmid. The sequence of the internal control probe is: CCCTGACCTACGGCGTGCAGTGCT (SEQ ID NO: 21). And the internal control probe has a HEX fluorescent label conjugated to its 5′ end and an BHQ1 quencher conjugated to its 3′ end.

### Example 2

This example evaluates the specificity of the primer-probe combination provided in example 1. Specificity refers to the ability to unequivocally detect the target nucleic acid present in a test sample, encompassing two key aspects: mycoplasma nucleic acids must be detectable, while non-mycoplasma nucleic acids must not be detected.
1. Validation of the detection range.

### (1) Materials.

\textcircled{1} Mycoplasma qPCR quantitative standards purchased from Minerva Biolabs: M. orale quantitative standard (Cat. 52-0112), M. pneumoniae quantitative standard (52-0119), A. laidlawii quantitative standard (52-0116), S. citri quantitative standard (52-0164), M. arginini quantitative standard (52-0129), M. fermentans quantitative standard (52-0117), M. hyorhinis quantitative standard (52-0130), M. synoviae quantitative standard (52-0124), M. gallisepticum quantitative standard (52-0115), and M. salivarium quantitative standard (52-0103).
\textcircled{2} The qPCR master mix was purchased from Nanjing Vazyme Biotech Co., Ltd.
\textcircled{3} Standard Solution: Each mycoplasma qPCR quantitative standard purchased from Minerva Biolabs was reconstituted and serially diluted to concentrations ranging from 5×10⁴ to 5×10⁻¹ genomic copies/pl to serve as points for the standard curve.
   (2) qPCR Reaction Mixture and Program.

The qPCR reaction mixture was prepared according to table 4.

TABLE 4

| | | |
|---|---|---|
| qPCR master mix | 12.5 | μl |
| Forward Primer (25 μM each) | 1 | μl |
| Reverse Primer (25 μM each) | 0.65 | μl |
| Detection Probe (25 μM each) | 0.4 | μl |
| Internal Control Probe (25 μM each) | 0.1 | μl |
| Test Template | 10 | μl |
| H₂O | 0.35 | μl |

The qPCR program was as follows: 37° C. for 2 minutes; 95° C. for 5 minutes; followed by 45 cycles of 95° C. for 15 seconds, 53° C. for 25 seconds, and 72° C. for 20 seconds, with plate reading after each cycle.

### (3) Results.

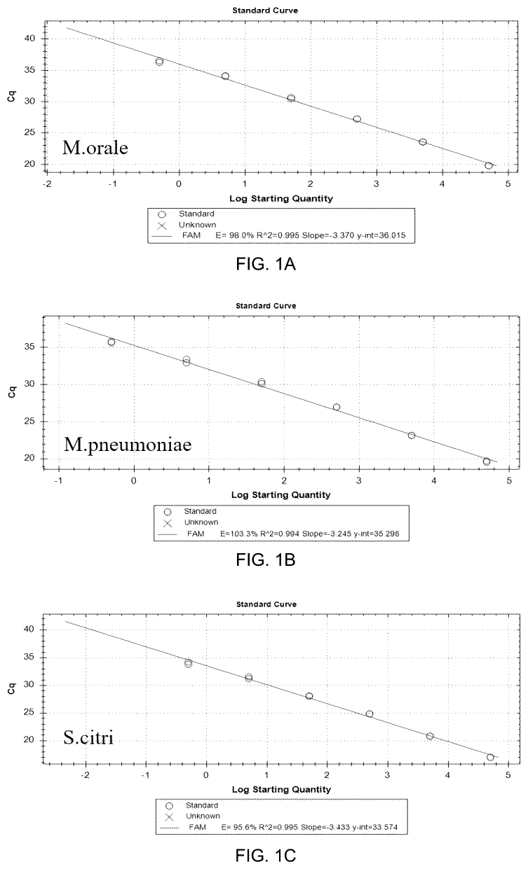

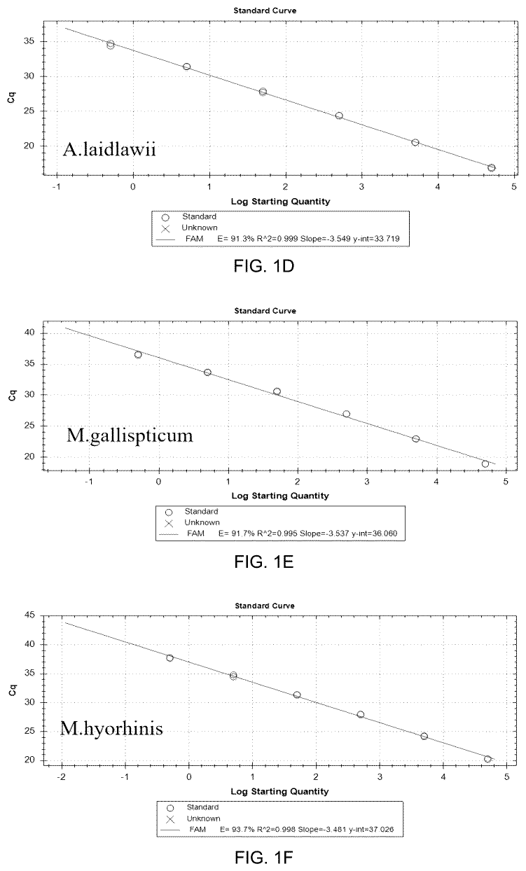

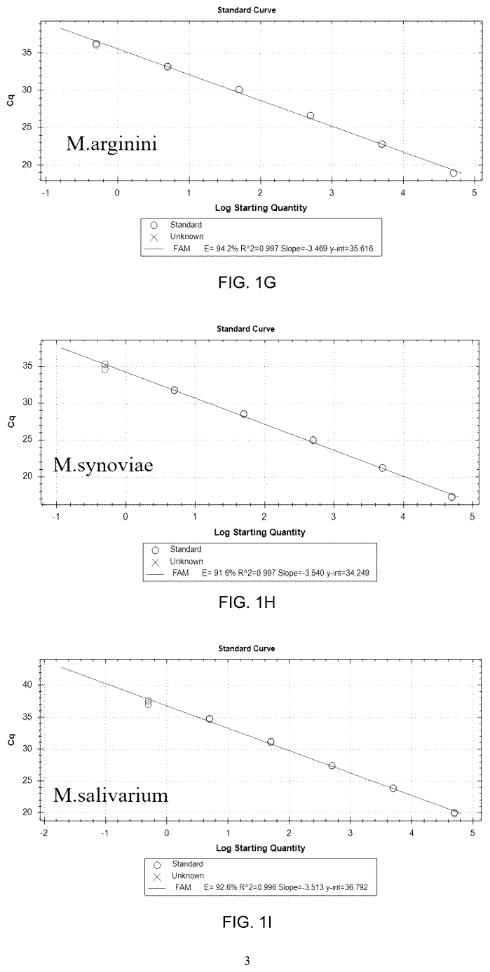

The detection results for the 10 mycoplasma standard samples in this example are shown in FIGS. 1A 1J. Across the dilution range of 5×10⁴ to 5×10⁻¹ genomic copies/μl, all mycoplasma species exhibited amplification efficiencies greater than 90% and R² values greater than 0.99.

Additionally, this example validated the artificially constructed 16S rRNA standard plasmids for M. pirum, M. mycoides, M. bovis, M. hominis, and U. urealyticum, as well as the genomic DNA of M. arthritidis, M. genitalium, and M. penetrans purchased from Minerva Biolabs. All tested mycoplasma species exhibited amplification efficiencies greater than 90% and R² values greater than 0.99 (data not shown).
2. Verification of cross-reactivity with non-mycoplasma species.

### (1). Materials.

Genomic DNA was extracted from the following bacterial species using the TaKaRa MiniBEST Bacteria Genomic DNA Extraction Kit Ver.3.0: Staphylococcus aureus, Clostridium sporogenes, Bacillus subtilis, Mycobacterium phlei, Pseudomonas aeruginosa, Escherichia coli, Micrococcus luteus, Salmonella Paratyphi, Streptococcus pyogenes, Bacillus cereus, Enterolysin yersinia, Staphylococcus epidermidis, and Corynebacterium diphtheriae.

Genomic DNA of Clostridium acetobutylicum, Streptococcus pneumoniae, and Lactobacillus acidophilus was purchased from Minerva Biolabs with product codes 51-0792, 51-0566, and 51-1723, respectively.

Genomic DNA of Aspergillus niger and Candida albicans was extracted using the SP Fungal DNA Kit D5542 (OMEGA, Cat. No. D5542-01).

Genomic DNA was extracted from human umbilical cord mesenchymal stem cells (MSCs), human embryonic kidney cells (HEK293), porcine kidney cells (PK-15), bovine kidney cells (MDBK), African green monkey kidney cells (Vero), Chinese hamster ovary cells (CHO-K1), and mouse subcutaneous connective tissue cells (A9) using the QIAamp DNA Mini Kit (50) (QIAGEN, Cat. No. 51304).

The genomic DNA of human Ad5 virus and AAV2 virus was obtained.

### (2) Preparation of Genomic DNA at Working Concentration.

The concentration of the extracted bacterial, fungal, and cellular genomic DNA was measured using a NanoDrop spectrophotometer. Based on the measured concentrations, the bacterial and fungal genomic DNA was diluted with RNase-free water to 1 ng/μl, respectively, while the human and animal cellular genomic DNA was diluted to 20 ng/μl, respectively. The genomic DNA of Clostridium acetobutylicum, Streptococcus pneunoniae, and Lactobacillus acidophilus, with an initial concentration of 10 ng per tube, was diluted to a working solution of 0.01 ng/μl, respectively. The genomic DNA of human Ad5 virus and AAV2 virus was quantified and subsequently diluted to 5×10⁶ copies/μl. The qPCR reaction mixture and program were consistent with those described in part 1, step (2).

### (3) Results.

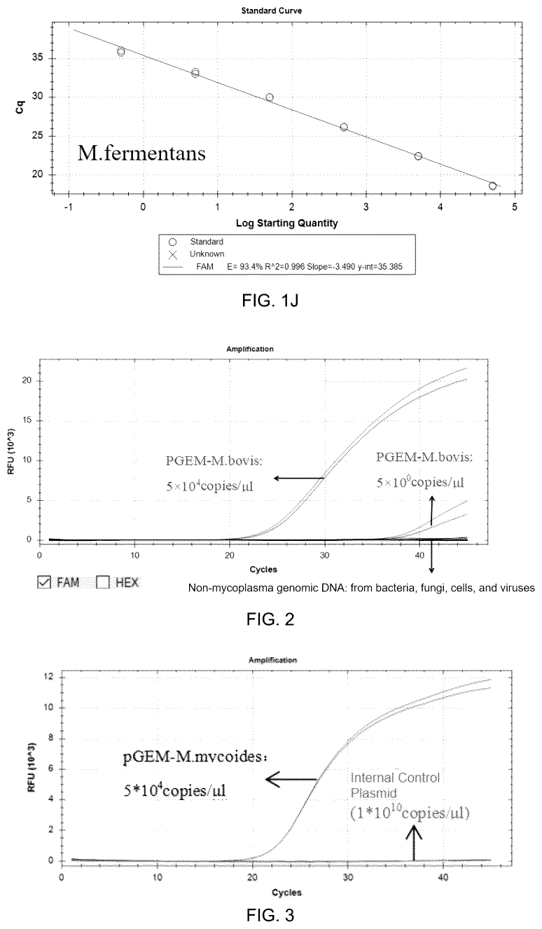

In repeated validation experiments, no amplification peak was observed in the NTC. Furthermore, as shown in FIG. 2, the primer-probe combination provided by the present disclosure exhibited no cross-reactivity with the genomic DNA of bacteria, fungi, human and animal cells, or common viruses, demonstrating excellent specificity. A summary of the amplification results for non-mycoplasma genomic DNA is presented in table 5.

TABLE 5

| | | | Human or | |
|---|---|---|---|---|
| Bacteria Listed | | | Animal Cell | Viruse |
| in EP or JP (0.1 | Other Bacteria (10 | Fungi (10 | (200 | (5 × 10⁷ |
| ng/reaction) | ng/reaction) | ng/reaction) | ng/reaction) | copies/reaction) |
| Clostridium | Clostridium sporogenes, | Aspergillus | MSC | Ad5 |
| acetobutylicum, | Staphylococcus aureus, | niger, | HEK293 | AAV2 |
| Streptococcus | Bacillus subtilis, | Candida | PK15 | |
| pneumoniae, | Mycobacterium phlei, | albicans | MDBK | |
| Lactobacillus | Pseudomonas aeruginosa, | | Vero | |
| acidophilus | Escherichia coli, Micrococcus | | CHO-K1 | |
| | luteus, Streptococcus | | A9 | |
| | pyogenes, Bacillus cereus, | | | |
| | Staphylococcus epidermidis, | | | |
| | Enterolysin yersinia, | | | |
| | Corynebacterium diphtheria, | | | |
| | Salmonella paratyphi | | | |

Determined CT: NA (indicating no cross-reactivity)

### Example 3

This example describes the validation of the internal control plasmid and its corresponding probe provided in example 1.
1. Verification of cross-reactivity between the primer-probe combination and the internal control plasmid.

Based on the size and measured concentration of the internal control plasmid, it was diluted to 1×10¹⁰ copies/μl and used as the reaction template.

Detection was performed using the qPCR reaction mixture and program described in example 2. The results, as shown in FIG. 3, demonstrate that the primer-probe combination exhibits no cross-reactivity with the internal control plasmid (1×10¹⁰ copies/μl).
2. Specificity validation of the primers and probe for the internal control plasmid.

### (1) Materials.

\textcircled{1} The genomic DNA of the bacterial, fungal, human, and animal cell species used was identical to that described in example 2.
\textcircled{2} Genomic DNA of M. orale, M. pneumoniae, and Spiroplasma helicoides was extracted using the TaKaRa MiniBEST Bacteria Genomic DNA Extraction Kit Ver.3.0 and quantified to a concentration of 1 ng/μl. Additionally, qPCR quantitative standards for Acholeplasma laidlawii and S. citri were purchased from Minerva Biolabs and diluted to 1×10⁵ copies/reaction, respectively.
   (2) The qPCR Reaction Mixture and Program were Identical to Those Described in Example 2.

### (3) Results.

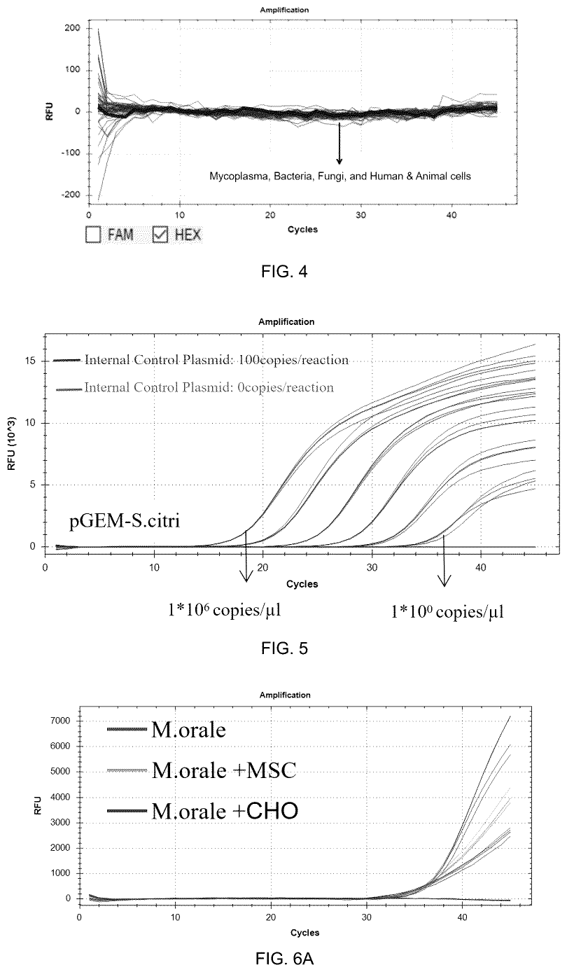

As shown in FIG. 4, the primers and probe for the internal control plasmid exhibited no cross-reactivity with the genomic DNA of bacteria, fungi, human and animal cells, or mycoplasma, demonstrating excellent specificity.
3. Effect of internal control plasmid addition on the mycoplasma detection system.

### (1) Materials.

\textcircled{1} Based on the size and measured concentration of the internal control plasmid, it was diluted to a concentration of 1×10³ copies/μl.
\textcircled{2} The 16S ribosomal RNA gene sequence of S. citri strain R8A2HP (GenBank: NR_036849.2) was selected as the target for the construction of a standard plasmid. Based on the plasmid size and measured concentration, the plasmid was serially diluted in 10-fold increments from an initial concentration of 1×10⁶ copies/μl down to 1×10¹ copies/μl to serve as templates for the standard curve.
   (2) The qPCR Reaction Mixture was Prepared According to Table 6, and the Program was Identical to Those Described in Example 2.

TABLE 6

| | | |
|---|---|---|
| qPCR master mix | 12.5 | μl |
| Forward Primer (25 μM each) | 1 | μl |
| Reverse Primer (25 μM each) | 0.65 | μl |
| Detection Probe (25 μM each) | 0.4 | μl |
| Internal Control Probe (25 μM each) | 0.1 | μl |
| pGEM-S. citri Standard Curve Template | 10 | μl |
| Internal Control Plasmid (1 × 10³ or 0 copies/μl) | 0.1 | μl |
| H₂O | 0.25 | μl |

### (3) Results.

In the present invention, the primers for the internal control plasmid are one of the primer pairs disclosed in example 1, and this specific pair is identical to the primer pair targeting Spiroplasma. Therefore, Spiroplasma serves as the optimal validation template. As shown in FIG. 5, the addition of the internal control plasmid at 150 copies/reaction had no impact on the Ct value or amplification efficiency of the original pGEM-S. citri reaction. It can therefore be inferred that it similarly does not affect the amplification of other mycoplasma species, indicating that this internal control plasmid is suitable for use in the mycoplasma detection of the present invention.

### Example 4

This example describes the validation of the LOD. The LOD is defined as the lowest concentration of the target mycoplasma or nucleic acid in a sample that can be detected. Verification at set concentrations requires only positive/negative identification and does not necessitate precise quantification. From a statistical perspective, the LOD is the lowest concentration of mycoplasma or copy number that yields a positive result in 95% of the replicate tests.

### 1. Validation Method.

The qPCR quantitative standards for the 10 mycoplasma species (identical to those in example 2) were serially diluted to concentrations of 100, 10, 2, 1, 0.5, 0.2, and 0.1 copies/μl. Three independent qPCR experiments were performed on three separate days, with each dilution replicated in at least 8 wells per experiment. The reaction mixture and program were consistent with those described in example 2.

### 2. Results.

As shown in table 7, the limits of detection were as follows: 0.5 copies/μl (equivalent to 5 copies/reaction) for M. orale; 0.5 copies/μl (5 copies/reaction) for M. pneumoniae; 0.2 copies/μl (2 copies/reaction) for S. citri; 0.1 copies/μl (1 copy/reaction) for A. laidlawii; 0.2 copies/μl (2 copies/reaction) for M. gallisepticum; 0.5 copies/μl (5 copies/reaction) for M. hyorhinis; 0.1 copies/μl (1 copy/reaction) for M. arginini; 0.1 copies/μl (1 copy/reaction) for M. synoviae; 0.1 copies/μl (1 copy/reaction) for M. fermentans; and 0.5 copies/μl (5 copies/reaction) for M. salivarium. These results demonstrate that the primer-probe combination provided by the present invention exhibits high sensitivity, with a detection limit ranging from 0.1 to 0.5 copies/μl.

TABLE 7

| | Positive/Total (Positivity Rate) | | | | | | |
|---|---|---|---|---|---|---|---|
| Mycoplasma | 100 copies/μl | 10 copies/μl | 2 copies/μl | 1 copies/μl | 0.5 copies/μl | 0.2 copies/μl | 0.1 copies/μl |
| M. orale | 24/24 | 24/24 | 24/24 | 24/24 | 24/24 | 21/24 | 17/24 |
| | (100.00%) | (100.00%) | (100.00%) | (100.00%) | (100.00%) | (87.50%) | (70.83%) |
| M. pneumoniae | 24/24 | 24/24 | 24/24 | 31/32 | 24/24 | 17/24 | 19/24 |
| | (100.00%) | (100.00%) | (100.00%) | (96.88%) | (100.00%) | (70.83%) | (79.17%) |
| S. citri | 24/24 | 24/24 | 24/24 | 24/24 | 24/24 | 23/24 | 20/24 |
| | (100.00%) | (100.00%) | (100.00%) | (100.00%) | (100.00%) | (95.83%) | (83.33%) |
| A. laidlawii | 24/24 | 24/24 | 24/24 | 24/24 | 24/24 | 24/24 | 24/24 |
| | (100.00%) | (100.00%) | (100.00%) | (100.00%) | (100.00%) | (100.00%) | (100.00%) |
| M. gallispticum | 24/24 | 24/24 | 24/24 | 24/24 | 24/24 | 23/24 | 17/24 |
| | (100.00%) | (100.00%) | (100.00%) | (100.00%) | (100.00%) | (95.83%) | (70.83%) |
| M. hyorhinis | 24/24 | 24/24 | 24/24 | 24/24 | 24/24 | 20/24 | 19/24 |
| | (100.00%) | (100.00%) | (100.00%) | (100.00%) | (100.00%) | (83.33%) | (79.17%) |
| M. arginini | 24/24 | 24/24 | 24/24 | 24/24 | 24/24 | 24/24 | 24/24 |
| | (100.00%) | (100.00%) | (100.00%) | (100.00%) | (100.00%) | (100.00%) | (100.00%) |
| M. synoviae | 24/24 | 24/24 | 24/24 | 24/24 | 24/24 | 24/24 | 24/24 |
| | (100.00%) | (100.00%) | (100.00%) | (100.00%) | (100.00%) | (100.00%) | (100.00%) |
| M. fermentans | 24/24 | 24/24 | 24/24 | 24/24 | 24/24 | 24/24 | 24/24 |
| | (100.00%) | (100.00%) | (100.00%) | (100.00%) | (100.00%) | (100.00%) | (100.00%) |
| M. salivarium | 24/24 | 24/24 | 24/24 | 24/24 | 24/24 | 21/24 | 21/24 |
| | (100.00%) | (100.00%) | (100.00%) | (100.00%) | (100.00%) | (87.50%) | (87.50%) |

### Example 5

To demonstrate that the solution of the present disclosure can replace the traditional pharmacopoeial methods, a comparability study on sensitivity must be conducted for the proposed method. The european pharmacopoeia (EP 10.0 2.6.7) stipulates that, (1) if a NAT method is to replace the mycoplasma culture method, it must be demonstrated that the LOD is at least ≤10 CFU/ml; (2) if a NAT method is to replace the indicator cell culture method, the LOD for each mycoplasma species tested must be at least 5100 CFU/ml.

Two approaches can be used for the comparability study: (1) perform the nucleic acid test and the pharmacopoeial method in parallel, detecting and analyzing the detection rate at the mycoplasma LOD concentration; or (2) compare the data from the NAT against an officially certified sensitivity standard with well-documented calibration and stability that has been validated for the pharmacopoeial method. This example adopts the second approach, using the 10 CFU sensitivity standard purchased from Minerva Biolabs to validate comparability.

### 1. Materials.

(1) 10 CFU standards purchased from Minerva Biolabs: 10 CFU A. laidlawii (102-8003), 10 CFU M. arginini (102-1003), 10 CFU M. fermentans (102-6003), 10 CFU M. gallisepticum (102-3003), 10 CFU M. hyorhinis (102-7003), 10 CFU M. orale (102-2003), 10 CFU My. pneumoniae (102-4003), 10 CFU M. synoviae (102-5003), 10 CFU S. citri (102-9003), 10 CFU M. salivarium (102-1103).
(2) QIAamp MinElute Virus Spin Kit (57704).
(3) The internal control plasmid was diluted to 300 copies/μl using RNase-free water.

### 2. Method.

(1) Add 1 ml of 5% FBS DMEM to each 10 CFU standard and its corresponding negative control. After thorough resuspension, aliquot the mixture into 200 μl portions per tube (1 ml can be aliquoted into 5 tubes). The aliquots can either be processed for extraction immediately or stored at −80° C.
(2) To each 200 μl aliquot of the 10 CFU sensitivity standard and the negative control, add 5 μl of the 300 copies/μl internal control plasmid. Subsequently, extract the nucleic acids using the QIAamp MinElute Virus Spin Kit. Elution is performed using 100 μl of RNase-free water (preheated to 65° C.), then reload the eluate onto the same spin column and perform a second elution step.
(3) qPCR verification was performed using the reaction mixture and program described in Example 2. A minimum of three independent extraction and qPCR experiments were conducted on separate days, involving a total of 6 extracted tubes. Each extracted sample was tested in 4 replicate qPCR wells, resulting in a total of 24 replicate data points. The acceptance criterion required at least 23 of the 24 replicates to be positive, corresponding to a positivity rate of 95%.

### 3. Results.

During the experimental procedure of this example, no amplification signal was detected in either the NC or the NTC. As shown in table 8, a 100% detection rate was achieved for all standards, with Ct value ranging from 26.66 to 36.18. These results demonstrate that the mycoplasma detection sensitivity of the present disclosure is ≤10 CFU/ml, supporting its suitability as a replacement for the pharmacopoeial culture method.

TABLE 8

| 10 CFU/ml | | Positivity | | | |
|---|---|---|---|---|---|
| Standards | Positive/Total | Rate | Mean Ct | SD | CV |
| M. orale | 24/24 | 100% | 35.98 | 0.29 | 0.81% |
| N. pneumoniae | 24/24 | 100% | 32.89 | 0.33 | 1.02% |
| S. citri | 24/24 | 100% | 26.66 | 0.15 | 0.57% |
| A. laidlawii | 24/24 | 100% | 31.96 | 0.16 | 0.51% |
| M. gallispticum | 24/24 | 100% | 34.46 | 0.22 | 0.65% |
| N. hyorhinis | 24/24 | 100% | 36.15 | 0.37 | 1.01% |
| M. arginini | 24/24 | 100% | 35.82 | 0.23 | 0.64% |
| N. synoviae | 24/24 | 100% | 34.10 | 0.19 | 0.56% |
| M. fermentans | 24/24 | 100% | 36.18 | 0.27 | 0.75% |
| N. salivarium | 24/24 | 100% | 36.43 | 0.38 | 1.04% |

### Example 6

Robustness refers to the ability of an analytical procedure to remain unaffected by small, deliberate variations in method parameters. For mycoplasma samples, such as those derived from cell culture, even after pre-treatment, residual host cells or their DNA may persist. These non-specific sequences can potentially interfere with the amplification of the mycoplasma target, thereby impacting the analytical sensitivity, making verification necessary. Therefore, this example validates the robustness of the detection method provided by the present disclosure.

### 1. Validation Method.

(1) MSCs and CHO cells were trypsinized and counted. A total of 1×10⁶ MSCs or CHO cells were resuspended in a mixture containing 200 μl of the 10 CFU/ml mycoplasma standard and 5 μl of the 300 copies/μl internal control plasmid (identical to that used in example 5). Concurrently, two control samples were prepared: a 10 CFU/ml mycoplasma control without added cells, and a cell control without added mycoplasma. Nucleic acids from all samples were then extracted using the QIAamp MinElute Virus Spin Kit.
(2) qPCR verification was performed using the reaction mixture and program described in example 2, with each sample tested in 4 replicate wells.

### 2. Results.

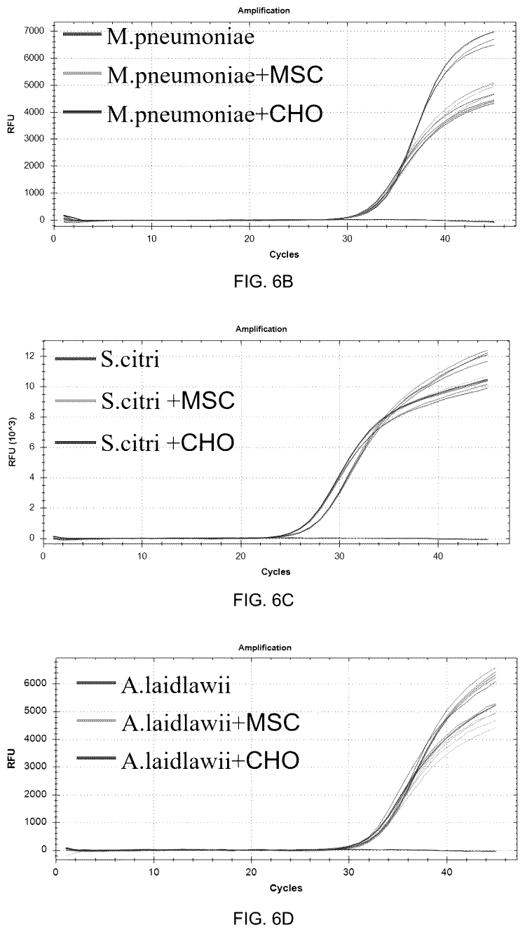

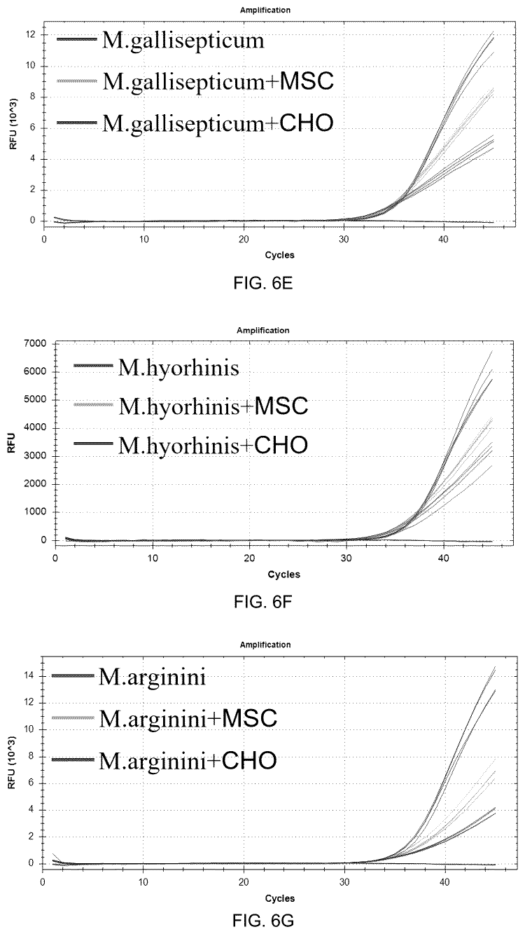

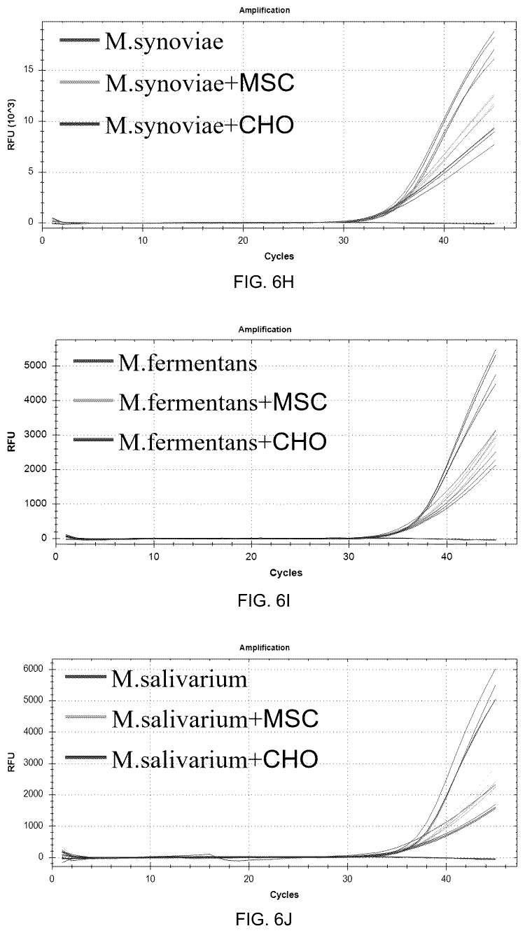

During the experimental procedure of this example, no amplification signal was detected in either the NC or the NTC. As shown in FIGS. 6A-6J, the presence of 1×10⁶ cells (MSCs/CHO) caused varying degrees of fluorescence suppression in the amplification curves of the different 10 CFU mycoplasma standards. However, this suppression did not affect the final qualitative interpretation (positive/negative call). Moreover, in actual sample testing, a centrifugation step is typically included to remove cells. The statistical results of the Ct values presented in table 9 further confirm that 1×10¹ cells (MSCs/CHO) do not significantly impact the amplification Ct values of the different 10 CFU mycoplasma standards.

TABLE 9

| Mycoplasma | | +1 × 10⁶MSC | | +1 × 10⁶CHO | |
|---|---|---|---|---|---|
| species | NC | Cells | ΔCq | Cells | ΔCq |
| 10 CFU A. laidlawii | 31.83 | 31.58 | 0.25 | 31.24 | 0.59 |
| Cq(n = 4) | (31.68-32.05) | (31.21-31.79) | | (31.18-31.34) | |
| 10 CFU M. hyorhinis | 35.10 | 34.27 | 0.83 | 34.60 | 0.49 |
| Cq (n = 4) | (35.00-35.22) | (34.06-34.45) | | (34.13-35.23) | |
| 10 CFU M. fermentans | 35.86 | 35.80 | 0.06 | 35.89 | −0.03 |
| Cq(n = 4) | (35.71-35.99) | (35.60-35.94) | | (35.16-36.33) | |
| 10 CFU M. arginini | 34.91 | 35.44 | −0.53 | 36.12 | −1.21 |
| Cq(n = 4) | (34.68-35.25) | (35.05-35.68) | | (35.94-36.32) | |
| 10 CFU M. gallisepticum | 34.34 | 33.81 | 0.54 | 33.64 | 0.70 |
| Cq(n = 4) | (34.21-34.44) | (33.68-34.01) | | (33.52-33.84) | |
| 10 CFU M. synoviae | 33.43 | 33.14 | 0.29 | 33.01 | 0.42 |
| Cq(n = 4) | (32.85-33.82) | (33.09-33.28) | | (32.78-33.30) | |
| 10 CFU S. citri | 25.61 | 24.65 | 0.95 | 24.64 | 0.97 |
| Cq(n = 4) | (25.50-25.69) | (24.42-24.78) | | (24.63-24.65) | |
| 10 CFU M. orale | 35.34 | 34.80 | 0.53 | 34.88 | 0.45 |
| Cq(n = 4) | (35.10-35.58) | (34.71-35.01) | | (34.43-35.51) | |
| 10 CFU M. pneumoniae | 32.93 | 31.78 | 1.15 | 31.96 | 0.97 |
| Cq(n = 4) | (32.85-33.01) | (31.69-31.86) | | (31.83-32.16) | |
| 10 CFU M. salivarium | 36.01 | 36.30 | −0.29 | 36.06 | −0.05 |
| Cq(n = 4) | (35.52-36.25) | (35.83-36.49) | | (34.56-36.75) | |

### Example 7

This example utilized the SilvaTestPrime online software (https://www.arb-silva. de/search/testprime/) to predict the coverage range of the mycoplasma species detectable by the primer-probe combination of the present disclosure. The matched sequences were downloaded, from which entries labeled “Uncultured” or “Unidentified” were removed. After further eliminating duplicate entries, the remaining sequences constituted the target rRNA sequences. Finally, NCBI Primer-BLAST was used to search for any potential accidental matches to non-rRNA sequences. The search and alignment results confirmed that this primer-probe combination can detect at least 132 species and 410 strains of mycoplasma. Table 10 summarizes the matching species and strains within the class Mollicutes.

TABLE 10

| Mollicutes | NO. of Species | NO. of Strain |
|---|---|---|
| Mycoplasma sp. | 101 | 328 |
| Spiroplasma sp. | 14 | 35 |
| Ureaplasma sp. | 5 | 28 |
| Entomoplasma sp. | 4 | 5 |
| Acholeplasma sp. | 8 | 14 |
| Total | 132 | 410 |

### Example 8

This example used a CHO cells culture as the test sample and applied the method of the present disclosure for mycoplasma detection. Furthermore, this example adopted the approach of adding the internal control plasmid directly to the test sample. The method comprises the following steps:

### (1) Sample Preparation and Nucleic Acid Extraction.

Transfer 1 ml of cells culture suspension to a 1.5 ml EP tube. Centrifuge at 500×g for 5 minutes to pellet the cells. Transfer 1 ml of the resulting cell-free supernatant to a new 1.5 ml EP tube. Centrifuge at 20,000×g for 10 minutes at 4° C. to pellet and enrich the mycoplasma. Carefully aspirate the supernatant without disturbing the pellet (approximately 20-30 μl of supernatant may be retained to avoid accidental loss of the pellet). Resuspend the mycoplasma pellet in 170 μl of 0.9% sterile NaCl solution. Use this resuspension to resuspend the previously pelleted cells (approximately 10^{A}6 cells). Finally, add 5 μl of the internal control plasmid to prepare the test solution for subsequent use.

Combine 195 μl of 0.9% sterile NaCl solution with 5 μl of the internal control plasmid to prepare NEC.

For the test solution and NEC obtained above, nucleic acids were extracted using the QIAamp MinElute Virus Spin Kit (QIAGEN, 57704). Elution was carried out with 100 μl of RNase-free water (preheated to 65° C.), then the eluate was reloaded onto the same spin column for a second elution step. This process yielded the sample template and the negative control template, respectively.

Combine 0.5 μl of the positive control genome, 0.5 μl of the internal control plasmid, and 9 μl of nuclease-free water to prepare PC.

Combine 0.5 μl of the internal control plasmid with 9.5 μl of nuclease-free water to prepare NTC.

(2) Preparation of PCR Tubes (Perform this Procedure on a Cooling Ice Box).

Test sample tube: Combine 15 μl of qPCR reaction mix with 10 μl of the sample template in a reaction tube. Prepare 2 replicates.

NEC tube: Combine 15 μl of qPCR reaction mix with 10 μl of the negative control template in a reaction tube. Prepare 2 replicates.

PC tube: Combine 15 μl of qPCR reaction mix with 10 μl of PC in a reaction tube. Prepare 2 replicates.

NTC tube: Combine 15 μl of qPCR reaction mix with 10 μl of NTC in a reaction tube. Prepare 2 replicates.

Create a dual-channel setup for FAM and HEX on the real-time PCR instrumen, then perform qPCR. The qPCR reaction conditions were as follows: 37° C. for 2 minutes; 95° C. for 5 minutes; followed by 45 cycles of 95° C. for 15 seconds, 53° C. for 25 seconds, and 72° C. for 20 seconds.

### (3). Perform Result Interpretation Based on the Ct Value.

Select the Ct value reading mode according to the specific qPCR instrument and its accompanying control software. Using the BIO-RAD CFX96 as an example, choose the “Regression” mode for the Cq Determination Mode, or alternatively, select the “Single Threshold” mode and manually drag the threshold line to the starting point of the linear amplification phase of the positive control. Record the Ct values for the test sample, NEC, PC, and NTC. The expected results of control samples should correspond to those outlined in table 2, and interpretation of the test sample results is based on the principles given in table 3.

If the internal control plasmid is added prior to nucleic acid extraction, the difference in Ct values between the internal control channel (HEX channel) in a mycoplasma-negative sample (FAM channel: no Ct value) and the NEC should be within ±3 cycles. If the internal control plasmid is added during PCR setup, the difference in Ct values between the internal control (HEX channel) in a mycoplasma-negative sample (FAM channel: no Ct value) and NEC must be within ±2 cycles. The detection results for this example are shown in table 11, indicating the absence of mycoplasma contamination in the CHO cells culture.

TABLE 11

| Samples | FAM channel | | HEX channel | | Result |
|---|---|---|---|---|---|
| PC | 33.64 Ct | 34.13 | 34.32 Ct | 33.87 | Mycoplasma |
| NEC | NA | NA | 35.02 Ct | 34.93 | Negative |
| NTC | NA | NA | 34.74 Ct | 35.11 | |
| Test Sample | NA | NA | 34.90 Ct | 35.25 | |

In summary, the present disclosure provides a primer-probe combination, a kit, and a method for the rapid detection of mycoplasma in biological samples. The method is characterized by its operational simplicity, short turnaround time, and low sample volume requirement. The primer-probe combination included in the kit offers broad coverage of mycoplasma species, high specificity, superior sensitivity, and strong robustness. Furthermore, the kit incorporates an internal control system to monitor extraction efficiency and potential PCR inhibition in samples, thereby preventing false-negative results. The kit of the present invention is suitable for the qualitative detection of mycoplasma contamination in biological products such as master cell banks, working cell banks, virus seed lots, as well as clinical cell therapy products and gene therapy products.

The above descriptions are only preferred embodiments of the present invention and are not intended to limit the present invention. Any modification, equivalent replacement, improvement, etc., made within the spirit and principle of the present invention shall be included in the protection of the present invention.

1. A primer-probe combination for rapid broad-spectrum detection of mycoplasma, comprising 10 forward primers, 5 reverse primers, and 4 detection probes; wherein the forward primers have the sequences as shown in SEQ ID NOs. 1-10, the reverse primers have the sequences as shown in SEQ ID NOs. 11-15, and the detection probes have the sequences as shown in SEQ ID NOs. 16-19; and the 5′ end of each detection probe is conjugated to a first fluorescent label and the 3′ end is conjugated to a quencher.

2. The primer-probe combination according to claim 1, wherein the first fluorescent label is selected from the group consisting of FAM, TET, NED, ROX, CY3, CY5, VIC, JOE, and HEX; and the quencher is selected from the group consisting of MGB, TAMRA, NFQ, ECLIPSE, DABCYL, BHQ1, and BHQ2.

3. The primer-probe combination according to claim 2, wherein the first fluorescent label is FAM, and the quencher is MGB.

4. A product for rapid broad-spectrum detection of mycoplasma, comprising the primer-probe combination according to any one of claims 1-3.

5. A kit for rapid broad-spectrum detection of mycoplasma, comprising the primer-probe combination according to any one of claims 1-3, and further comprising an internal control plasmid and a internal control probe for monitoring PCR inhibition during mycoplasma detection.

6. The kit according to claim 5, wherein the internal control plasmid comprises a target fragment having the sequence as shown in SEQ ID NO: 20, and the internal control probe has the sequence as shown in SEQ ID NO: 21; and wherein the 5′ end of the internal control probe is conjugated to a second fluorescent label that is different from the first fluorescent label, and the 3′ end is conjugated to a quencher.

7. The kit according to claim 5, wherein the kit further comprises a qPCR reaction mix, a positive control, and PCR-grade water.

8. The kit according to claim 7, wherein the qPCR reaction mix comprises a PCR buffer, dNTPs, a hot-start Taq polymerase, and MgCl₂; and the positive control is a buffer solution containing the genomic DNA of Mycoplasma pneumoniae and Mycoplasma orale.

9. A method for rapid broad-spectrum detection of mycoplasma, qualitatively detecting mycoplasma contamination in sampled specimens and biological products using the kit according to claim 5.

10. The method according to claim 9, comprising the following steps:
S1. prepare a test solution from the sample to be tested, and spike the internal control plasmid into either the test solution or the qPCR reaction mix; prepare a negative control by formulating the internal control plasmid in sterile saline; prepare a positive control by adding the positive control genome to a mixture of the internal control plasmid and nuclease-free water followed by dilution; prepare a no-template control by formulating the internal control plasmid in nuclease-free water; extract nucleic acids from the test solution and the negative control, respectively;
S2. on a real-time PCR instrument, create a dual-channel setup for the first and second fluorescent labels; perform qPCR separately for the test solution, negative control, positive control, and no-template control; record the Ct values for both channels from each reaction;
S3. interpret the results based on the Ct value:
a result is interpreted as mycoplasma positive if the Ct value in the mycoplasma detection channel is less than 40 and a normal amplification curve is present; as PCR inhibition if no Ct value is obtained in both the mycoplasma channel and the internal control channel; as mycoplasma negative if no Ct value is obtained in the mycoplasma channel while the Ct value in the internal control channel is less than 40 with a normal amplification curve; as an invalid result if the Ct value in the mycoplasma channel is 40 or greater with a normal amplification curve and the Ct value in the internal control channel is less than 40 with a normal amplification curve; and as PCR inhibition if the Ct value in the mycoplasma channel is 40 or greater with a normal amplification curve but no Ct value is obtained in the internal control channel.
